(19) United States
(12) Patent Application Publication
(10) Pub. No.: US 2025/0252286 A1
(43) Pub. Date: Aug. 7, 2025

(54) APPROXIMATING THE CLASSICAL PARTITION FUNCTION OF A MOLECULAR SYSTEM

(71) Applicant: SB Technology, Inc., Tarrytown, NY (US)

(72) Inventors: Martin Ganahl, Neuberg an der Muerz (AT); Maarten Van Damme, Humbeek (BE); Andrea Bortolato, Metuchen, NJ (US); Adam GM Lewis, Toronto (CA); Romelia del Carmen Salomon Ferrer, San Diego, CA (US)

(21) Appl. No.: 18/431,856

(22) Filed: Feb. 2, 2024

(51) Int. Cl. G06N 3/044

(52) U.S. Cl. CPC G06N 3/044; G16C 10/00; G16C 20/30

(57) ABSTRACT

Methods, systems, and apparatus, including computer programs encoded on a computer storage medium, for approximating the classical partition function of a molecular system. In one aspect, a system comprises receiving a set of inputs characterizing thermodynamic entities of a non-lattice system with one or more molecular degrees of freedom, approximating a partition function of the non-lattice system using a tensor network and based at least in part on the set of inputs, wherein approximating a partition function of the non-lattice system comprises using a molecular configuration sampler to vary over the one or more molecular degrees of freedom, and determining a system characterization entity from the partition function.

### BACKGROUND

This specification relates to the partition function of a molecular system, e.g., a system of a number of particles. A partition function provides the statistical properties of the system in thermodynamic equilibrium by describing the system of particles as an ensemble of all the possible energy states, e.g., energy levels, that the system can take, e.g., to quantify the behavior of the particles of the system. In particular, the partition function can be posed as an integral over a higher-dimensional probability distribution in which the function represents the summation of all the possible states in the system weighted by the probability of each state, e.g., a Boltzmann distribution. In the particular case of a Boltzmann distribution, each system state is associated with an energy.

Efficient methods for calculating partition functions are well-known for certain lattice-based systems and spin-systems, e.g., crystals and variants of the (quantum) Ising model. In general, calculating partition functions for many-body systems (classical or quantum) can require running a computationally-intensive molecular dynamics or Monte Carlo simulation.

### SUMMARY

This specification describes a system implemented as computer program(s) on one or more computers in one or more locations that can approximate the classical partition function of a molecular system. In this specification, a molecular system refers to a non-lattice system of molecules in which the structure of the partition function is unknown. As an example, the partition function can be for a system of one or more biological molecules, e.g., a protein and a small molecule known as a ligand.

The system can receive a set of inputs that characterize the thermodynamic state of a non-lattice system. For example, the set of inputs can include one or more molecular degrees of freedom(s), a force-field function, particle masses, and a temperature. The system can then process the inputs to construct a tensor network, e.g., a collection of one or more tensors, as an approximation to a high-dimensional probability distribution over the degrees of freedom of the system, e.g., a Boltzmann distribution that characterizes the probability of finding a particle in a specific energy state at a given temperature. The tensor network approximation can then be used to compute efficiently an approximation to the partition function of the original probability distribution, e.g., by integrating the probability function.

A tensor is an N-dimensional extension of a vector that constructs a multilinear map between the entries of each dimension using N indices. A tensor network includes one or more tensors that are associated together via operations, e.g., contractions defining a sum of products along the entries of each index in a specified pair of indices. In the particular implementation discussed, the tensor network approximation of the partition function is stored on one or more computers in one or more locations.

In particular, a tensor network can efficiently store a high-dimensional function by decomposing the high-dimensional function into a low-rank approximation, e.g., by contracting matrix product states, e.g., smaller tensors, to compress the set of values needed to express the original function. More specifically, a matrix product state approximation can approximately reconstruct the whole high-dimensional tensor when contracted, e.g., summed over all indices, while necessitating less computational memory than storing the entire high-dimensional tensor as an object in memory. In particular, the function can be decomposed into a matrix product state approximation such that evaluating a given multi-index of the matrix product state returns an approximation to the true value of the full tensor at the same multi-index. In particular, by approximating a probability distribution using a matrix product state approximation, it is possible to efficiently carry out operations on the function represented by the tensors, e.g., the computation of the partition function, by manipulating the matrix product state approximation.

The system can approximate the probability distribution by varying over the one or more molecular degrees of freedom using a molecular configuration sampler, e.g., to vary the configuration of the system over different possible combinations of translational and rotational degrees of freedom, and sampling indices to evaluate the exact values of the probability distribution of finding a particle in a specific energy state at a given temperature at the specific combination of values of the degrees of freedom. In particular, the system can sample an index for evaluation and perform a numerical or analytical computation of the probability function to yield an evaluated energy value at the index that can be incorporated into the tensor network approximation as an exact value. The system can then provide an estimate for the other values by interpolating the unevaluated values using the evaluated values, e.g., through the creation of a spline.

More specifically, the system can adaptively construct a grid over degrees of freedom configurations, e.g., an adaptive mesh grid 160, using an adaptive mesh constructor 150. The system can select regions of high importance, high variance, or both to the probability distribution of energy states at a given temperature over all allowable states to evaluate exactly and interpolate the other unevaluated values, e.g., the values on the grid 160. The system can determine regions within the phase space, e.g., a space representing all possible states of the probability distribution, where the tensor network approximation has the highest discrepancy with respect to the exact probability distribution and incorporate them into the tensor network approximation as exact values, e.g., using a tensor-train cross approximation algorithm. In particular, the probability distribution can be a Boltzmann distribution.

In particular, the system can sample states of the phase space and evaluate their exact probability, e.g., by performing an analytical or numerical computation, and can incorporate the evaluated probability into the tensor network approximation, over a number of iterations. For each iteration, the system can alternately optimize the values of two consecutive degrees of freedom to determine a multi-index, evaluate the probability of the determined multi-index, and compare the evaluated value to the approximated value provided by the tensor network, e.g., using interpolation. The system can then determine a pivot multi-index as the multi-index with the largest approximation error, e.g., the largest discrepancy between the evaluated probability and the probability provided by the tensor network, and incorporate the pivot multi-index into the tensor network approximation as an evaluated value.

The approximated probability distribution and the partition function, e.g., the normalizing constant of the probability distribution, can be used to determine molecular system characterization entities, such as one or more marginal and moment distributions over possible degrees of freedom of the partition function. Additionally, the partition function can be used to characterize solubility and to evaluate potential molecular structure and polymer conformations. In the particular example of approximating a partition function for a protein and ligand, the approximated partition function can be used to determine a binding free energy landscape to assess a distribution of possible docking configurations.

According to a first aspect there is provided a method for receiving a set of inputs characterizing thermodynamic entities of a non-lattice system with one or more molecular degrees of freedom, approximating a partition function of the non-lattice system using a tensor network and based at least in part on the set of inputs, wherein approximating a partition function of the system comprises using a molecular configuration sampler to vary over the one or more molecular degrees of freedom, and determining a system characterization entity from the approximated partition function.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on its software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Particular embodiments of the subject matter described in this specification can be implemented so as to realize one or more of the following advantages.

The techniques of this specification enable the approximation of a partition function in an efficient manner that reduces computational resources relative to standard approaches. In particular, the system can allow for approximating the partition function at polynomial cost in system size as opposed to the exponential cost generally required for the partition function's exact computation with computationally-intensive molecular dynamics or Monte Carlo simulation. Molecular dynamics or Monte Carlo simulation approaches are analogous to computing the values of the full tensor network. As an example, approximating the partition function of a non-lattice system using a tensor network to approximate the probability function requires less total function calls sampling, e.g., on the order of 10,000s compared to millions of energy function calls for molecular dynamic (MD) based standard methods. Additionally, storing the matrix product state as a low-rank approximation to the full tensor network probability distribution does not require as much computational memory as running the molecular dynamics simulation.

In particular implementations, the tensor network approximation is constructed using a tensor-train cross approximation. The techniques of this application augment the sampling approach, which is based on sampling discrete values from a grid along respective indices for each tensor dimension, to enable for continuous sampling of index values and the flexibility to update grid placement. This implementation uses an adaptive mesh grid to refine adaptively the resolution of states in different regions of phase space as required, e.g., with respect to the accuracy of the approximation. For example, areas of high probability, strong variations, or both, can be sampled at a finer resolution than areas with low probability, less variation, or both. In particular, this approximation technique can yield results in unsampled regions of phase space with potentially high fidelity.

Additionally, the techniques as described can be used to approximate the partition function of systems of any molecular configuration, including non-lattice and lattice-based systems, e.g., crystal structures, using the same approach. Additionally, the tensor network approximation does not require the gathering of experimental data to quantify the probability of one or more states of the phase space, thereby reducing experimental waste and the need for human resources.

Furthermore, the approximation of the partition function is constructed in a controlled manner that does not suffer from statistical error. In particular, the approximation does not require training, unlike machine learning approaches. Similarly, the approximation does not require verification of calculation convergence, unlike molecular dynamics methods and Monte Carlo methods which rely on gathering enough samples at different molecular conformations from the energy function. This allows the propagation of error to be limited when system characterization derivative quantities or functions, e.g., quantities or functions derived from the approximated partition function, are used for downstream use cases. As an example, the approximated partition function can be used for solubility prediction, e.g., computing the free energy of solvation for a given molecule and substrate, crystal structure prediction, e.g., predicting the most likely solid-state arrangements of a given molecule, and polymer conformation prediction, e.g., predicting the experimentally observed states of the molecule under different conditions. As another example, it can be used to inform the design of catalysts, active materials, and the removal of per- and polyfluorinated substances (“forever chemicals”) that can have serious health effects.

In some cases, the techniques of this specification can be combined with Monte Carlo or molecular dynamics simulation approaches to increase the speed of calculation. In particular, the method can be used to enhance existing MD-based computations of thermodynamic quantities by making more efficient use of the data generated by standard free energy perturbation (SFEP) calculations. To this end, the method can incorporate the samples generated during an MD or MCMC into an existing tensor-train approximation of the corresponding probability distribution, or train one from scratch.

Moreover, the approximation of the partition function can be used as an integrated solution for drug discovery. More specifically, the partition function can be used to compute simultaneously favorable conformations of a molecular system including a protein and ligand, e.g., a target and a drug. In particular, embodiments described in this specification can use the approximated partition function to predict accurately the binding affinity between molecules using the manipulation of the conformation of the molecular system. This enables the system to calculate potential poses and docking sites at the same time as binding affinities, which can allow for a more accurate prediction of how active a given molecule will be at treating a disease associated with the target, thereby accelerating the hit-to-lead and lead-optimization stages of drug discovery. Furthermore, the partition function for the target and the drug can be performed within minutes given a single modern GPU core, and is further scalable to millions of candidate drugs by employing cloud-based computer architectures.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a system diagram of an example non-lattice partition function approximation system that can approximate a partition function using a tensor network approximation.

FIG. 2 illustrates how a matrix product state can be used to approximate a full tensor.

FIG. 3 demonstrates the application of a tensor-train cross approximation algorithm as the tensor network approximation.

FIG. 4 depicts an example approximated probability function as a result of adaptive mesh sampling, e.g., by implementing the tensor-train cross approximation algorithm of FIG. 3.

FIG. 5 illustrates an example of the approximated partition function and the marginal distributions calculated for an example molecule with two degrees of freedom, e.g., using the non-lattice partition function approximation system of FIG. 1.

FIG. 6 demonstrates how an approximated partition function for a non-lattice molecular system including a protein and ligand can be used to assess the binding free energy landscape, e.g., as part of a drug discovery process.

FIG. 7 is a flow chart of operations for approximating a partition function of a non-lattice system using a tensor network.

FIG. 8 is a flow chart of operations for constructing the tensor network approximation of FIG. 7.

FIG. 9 is a flow chart of operations for adaptively evaluating an energy function to provide the tensor network approximation of FIG. 8.

Like reference numbers and designations in the various drawings indicate like elements.

### DETAILED DESCRIPTION

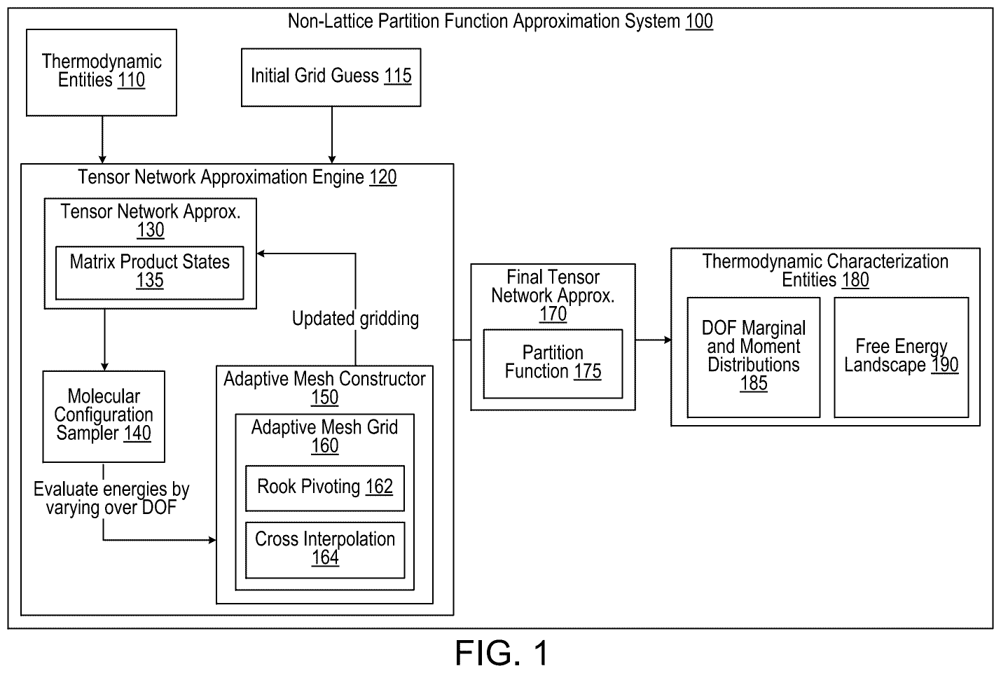

FIG. 1 depicts an example non-lattice partition function approximation system 100. The non-lattice partition function approximation system 100 is an example of a system implemented as computer program(s) on one or more computers in one or more locations in which the systems, components, and techniques described below are implemented.

The non-lattice partition function approximation system 100 can receive one or more inputs, e.g., the thermodynamic entities 110, to characterize the thermodynamic state of a non-lattice system of one or more molecules. As an example, a non-lattice system can be a molecular system such as a bio-molecular system, e.g., a protein and a ligand, or an inorganic molecular system, e.g., any non-crystalline solid, such as a glass, rubber, plastic, or gel. In particular, the thermodynamic entities 110 can characterize different thermodynamic aspects of the non-lattice system and can include one or more of molecular degrees of freedom, a molecular electric force-field function, particle masses, and temperature to characterize the various kinds of potential and kinetic energies of the non-lattice system.

In particular, the molecular degrees of freedom can specify one or more directions of available motion for the one or more molecules of the non-lattice system. As an example, the molecular degrees of freedom for a bio-molecular system, e.g., a protein and ligand, can include soft rotational degrees of freedom around certain bonds that can be twisted to provide different folding configurations. In particular, the molecular degrees of freedom for a protein can include Ramachandran angles, e.g., angles that describe the torsion of certain amino acids in the backbone of a protein. As another example, the molecular degrees of freedom can include degrees of freedom that describe the number of ways a gas can move, rotate, or vibrate in space. As yet another example, the molecular degrees of freedom can include degrees of freedom that describe the number of ways atoms within glass can move to fill vacancies within an amorphous structure. Glass can be considered to be an amorphous solid in certain respects.

The molecular electric force-field function can be calculated from the position and momenta of the particles in the system 100. In some cases, the molecular electric force-field function can be calculated using standard classical (non-quantum) methods. In a classical system, the molecular electric force-field function is given as the sum of the kinetic and potential energies of the constituent atomic nuclei:

$$E ( r , p ) = {\sum }_{i} \frac{{p}_{i}^{2}}{2 {m}_{i}} + V ( {r}_{1} , {r}_{2} , … , {r}_{N} ) ,$$

where i indexes over each of N atoms, pᵢ is the momentum of each atom, mᵢ is the mass of each atom, and V is a potential energy function, e.g., as defined by the electrostatic and van der Waals interactions between each of the atoms, parametrized by rᵢ, the distance between each of the atoms.

In particular, the molecular electric force-field function can be evaluated in analytical or numerical form. In the case of a simple molecular system, the system 100 can define a closed-form solution, e.g., using the equation above, for the potential energy function V. In the case of more complex molecular systems, e.g., molecular systems with more than one molecule, numerical methods can be used to calculate the potential energy as an integral over the charge density and potential function, e.g., over a defined volume of space. Furthermore, in the case of an evolving molecular system, such as a protein and ligand system in which the energy evolves over time as the molecules approach and begin to bind, numerical methods can be used to calculate and evolve the potential energy forward in time. In some cases, the molecular electric force-field function can be provided as a black-box model to the system 100, e.g., the system 100 can query values of the electric force-field function at different molecular configurations without evaluating the molecular force-field function directly.

The system 100 can process the input thermodynamic entities 110 along with an initial grid guess 115 using the tensor network approximation engine 120. In particular, the initial grid guess informs the initialization of the adaptive mesh grid 160 for each molecular degree of freedom. The adaptive mesh constructor 150 can then modify the adaptive mesh grid 160 over a sequence of iterations, as will be discussed in further detail below. In some cases, the grid 160 of allowable values can be discrete. In other cases, the grid 160 can be continuous, e.g., the grid can be expanded to values not previously incorporated into the grid. In the particular example depicted, the tensor network approximation engine 120 allows for continuous gridding for each of the variables specified by the molecular degrees of freedom.

The tensor network approximation engine 120 can construct a tensor network approximation 130 of the high-dimensional probability function that provides the probability of finding a particle in a specific energy state at a given temperature, e.g., the Boltzmann distribution, based on the thermodynamic entities 110 and the initial grid guess 115. As a helpful conceptualization, the high-dimensional probability distribution over possible degree of freedom configurations of a non-lattice system can be envisioned as a function whose values can be calculated at specific higher-dimensional points in accordance with the values of different axes, e.g., a higher dimensional analog to the way a function Y in the XY-plane can take values according to values of X. In this case, the probability function exists in a higher-dimensional space defined by the input variables as axes, e.g., the one or more molecular degrees of freedom each parametrize different axes along which the probability function can be evaluated.

These degrees of freedom axes can be considered as dimensions of the tensor that can be traversed by modifying the value of an index associated with each respective dimension of the tensor. Furthermore, the combination of values of the different axes can be considered as a multi-index of the tensor. In particular, a value of the probability distribution, e.g., a Boltzmann distribution, for the molecular system can be defined at a specific multi-index, e.g., a set of indices that provides index values for each dimension of the tensor. More specifically, a defined index value for each of the one or more degrees of freedom can provide a definite value of the probability function for the molecular system at the index values provided along each axis. In this case, the index values specify a certain conformation of the one or more molecules of the molecular system.

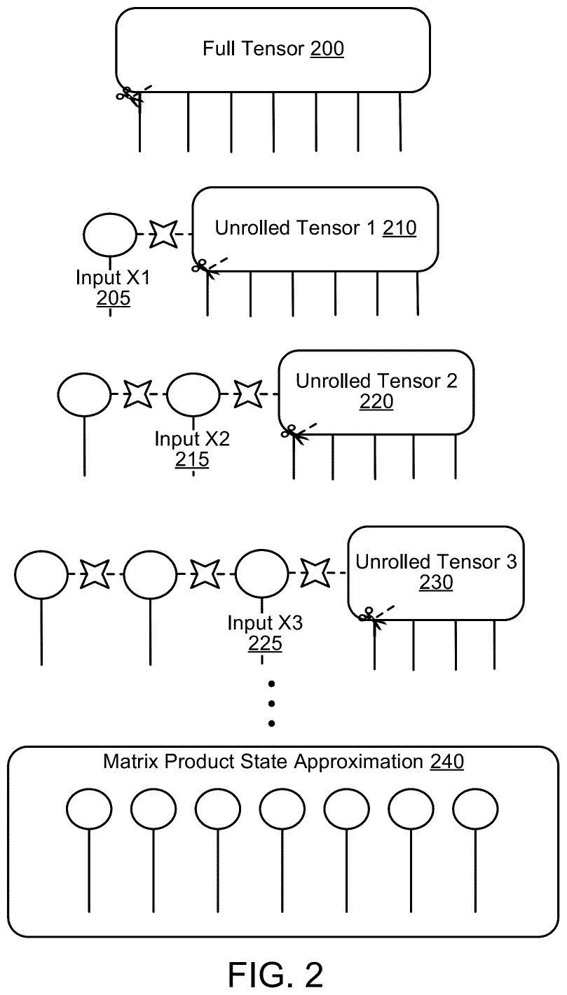

In particular, the grid or gridding for each degree of freedom variable defines the index values for which the particular variable can be initially evaluated in the tensor network, e.g., by calculating the probability function corresponding to the particular conformation specified by the degrees of freedom as indicated by a multi-index. The places where the values are defined according to the gridding are “knowable indices”, e.g., indices at which the probability function can be evaluated using a numerical or analytical computation for a given variable. More specifically, after discretizing each degree of freedom variable, every point in space can be associated with a value of the probability function, e.g., at every intersection of the gridding provided by each degree of freedom variable. However, the system 100 does not necessarily evaluate the probability function at each of the intersection points. Instead, the system 100 evaluates the probability function at a number of multi-indices that are chosen based on a discrepancy between the current tensor network approximation and the evaluation at the multi-index, as will be described below and in more detail in FIG. 2.

In some cases, the initial grid guess 115 can be based on a discretization scheme for each degree of freedom variable. In particular, gridding for each respective molecular degree of freedom can specify a spectrum of conformations for the molecular system over a range of angles or radians. In some cases, the spectrum of conformations for the molecular system can be defined on an evenly discretized grid, e.g., every N degrees where N can be 2, 5, or 15 degrees, or over a range of radians, e.g., every π/16, π/8, or π/4 radians, for each molecular degree of freedom. In other cases, different discretizations can be defined for each molecular degree of freedom. For example, the first molecular degree of freedom can be discretized every 5.5 degrees or 0.96 radians, the second degree of freedom can be discretized every 3 degrees or 0.52 radians, the third degree of freedom can be discretized every 16 degrees or 0.28 radians, etc.

As another example, the initial grid guess 115 can be random for each degree of freedom variable, e.g., the initial grid guess 115 can include a random seed that can be used as the seed for a pseudorandom number generator to generate the knowable indices of the variable in the tensor network approximation 130. As a further example, the initial grid guess 115 can be chosen by heuristics that are informed by one or more of the parameters of the system, e.g., the number of molecules, the molecular weight of the molecules, the number of small molecules with respect to the number of large molecules, etc. As yet another example, the initial grid guess 115 for some of the degree of freedom variables can be informed by one or more parameters of the system and the initial grid guess 115 for the rest of the variables can be generated randomly, e.g., using a pseudorandom number generator. In some cases, the initial grid guess 115 can start off with an initial discretization for each of the variables and become finer as required, e.g., using the adaptive mesh grid, which will be described in more detail below.

The tensor network approximation engine 120 can process the thermodynamic values 110 and the initial grid guess 115 to construct a tensor network approximation 130 of the probability function. In particular, the tensor network approximation engine 120 can initialize a set of one or more matrix product states 135 in accordance with the degrees of freedom, e.g., the molecular degrees of freedom of the molecular system. For example, there can be one matrix product state approximation for each molecule in the molecular system.

In particular, each leg of the tensor can represent a degree of freedom variable and therefore a traversable dimension of the tensor. As an example, the number of degrees of freedom can range from a few for simple, small molecules, to dozens for larger organic systems, to many hundreds or thousands for large biomolecules. For example, there can be two degrees of freedom for a simple system, 20 or 30 for a molecule, and hundreds for a system with two or more interacting molecules. As described above, each degree of freedom of the non-lattice system can be thought of as providing a traversable axis along which the probability function can be evaluated along a certain gridding, e.g., each degree of freedom provides a new dimension that characterizes a potential spectrum of molecular degree of freedom conformations that can be evaluated by the system 100 using a multi-index.

In the particular example depicted, the tensor network approximation engine 120 can use the molecular configuration sampler 140 to evaluate different energies of the molecular system by manipulating the one or more degrees of freedom. More specifically, the molecular configuration sampler 140 can manipulate the one or more degrees of freedom to change the conformation, e.g., the shape or structure, of the one or more molecules in the molecular system by varying the degrees of freedom and then determine whether or not to add one or more additional values of the probability function to the tensor network approximation 130. As an example, in the case of a protein system, the molecular configuration sampler 140 can vary over the angular and dihedral rotational degrees of freedom to change the conformation of the protein.

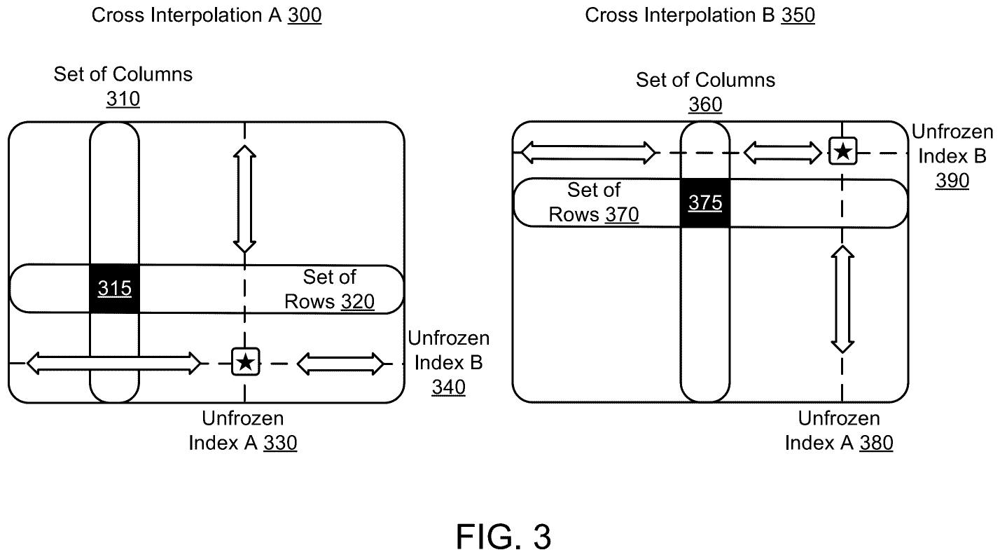

The different conformations of the molecular system are associated with different energy distributions, e.g., energy states of the probability function, and the different energies can impact the placement of the evaluated indices and the unevaluated indices of the adaptive mesh grid 160. In the particular example depicted, the adaptive mesh constructor 150 can use rook pivoting 162 and cross-interpolation 164 to adjust the placement of the adaptive mesh grid 160, e.g., the unevaluated multi-indices of the probability function for the tensor network approximation 130. In particular, the adaptive mesh constructor 150 can freeze, e.g., hold constant, all but two of the indices corresponding to the degree of freedom variables of the tensor network approximation 130 and alternatively vary the two unfrozen indices to identify a certain multi-index that maximizes a criterion as a pivot index. In this case, the pivot indices become evaluated values of the tensor network. An example of an adaptive mesh constructor 150 using rook pivoting 162 and cross-interpolation 164 to adaptively identify a number of multi-indices for inclusion into the tensor network approximation 130 as exact values will be covered in more detail in FIG. 3.

In particular, the adaptive mesh grid 160 controls the possible values of the pivot indices, e.g., indices that support the grid of unevaluated values that can be approximated with the tensor network approximation 130, e.g., using interpolation. In some cases, the two unfrozen indices can be varied and evaluated using discrete values, e.g., the values provided by the current gridding for the two unfrozen degrees of freedom. In other cases, the two unfrozen indices can be varied and evaluated using continuous values, e.g., the system 100 can alternatively vary each unfrozen index to identify a multi-index that is not currently present in the adaptive mesh grid 160. In particular, the adaptive mesh constructor 150 can modify the gridding for the two unfrozen indices in order to locate the best placement for the adaptive mesh grid 160, e.g., regardless of whether or not the best placement involves values specified by the current gridding for the two unfrozen degrees of freedom. In this case, the ability of the adaptive mesh grid 150 to accommodate continuous values enables the adaptive mesh constructor 150 to adaptively sample values of the probability function with high resolution in regions of importance, e.g., high probability, high variance, or both, for the probability function.

As an example, in the case that temperature, mass, electric force-field function, and five molecular degrees of freedom are included as processed thermodynamic values 110, the system 100 can vary two of the five molecular degrees of freedom using the molecular configuration sampler 140 and freeze the gridding for the other three degrees of freedom. The system 100 can then alternatively change the knowable values of the probability function in accordance with varying the values of the two unfrozen indices, e.g., either using random sampling or by evaluating values along the current gridding for each respective degree of freedom, to identify pivot-indices.

The system 100 can then add the identified pivot indices to the tensor network approximation 130 as multi-indices in which the probability function is evaluated exactly using a numerical or analytical computation. The other multi-index combinations of the tensor, e.g., the multi-indices that are on the unevaluated grid can be approximated from the known evaluated values, e.g., using an interpolation or regression method. In particular, the system 100 can fit a linear, quadratic, or spline approximation to interpolate the unevaluated indices of the probability function. The updated gridding, e.g., the adaptive mesh grid 160 provided by the adaptive mesh constructor 150, results in a corresponding change in the matrix product state approximation 135, e.g., by providing an exact evaluated value at one or more identified pivot indices. In particular, the evaluated values of the probability function at the pivot-indices can be incorporated at the corresponding multi-index of the matrix product state approximation 135 to improve the low-rank approximation of the high-dimensional probability function and, by extension, the partition function as an integral over the high-dimensional probability function.

The system 100 can adaptively refine the grid 160 of unevaluated indices over a number of iterations using the tensor network approximation engine 120, as represented by the cycle between the tensor network approximation 130, the molecular configuration sampler 140, and the adaptive mesh constructor 150. More specifically, the molecular configuration sampler 140 can evaluate energy probabilities by varying over the degrees of freedom in the molecular system, the adaptive mesh constructor 150 can update the adaptive mesh grid 160 according to the new conformation, and the updated gridding can cause a corresponding change in the matrix product state approximation 135.

In some examples, the number of iterations for the tensor network approximation engine 120 to approximate the tensor network approximation 120 is predefined. In other cases, the number of iterations can be arbitrary, but it is expected that the error of the approximation decreases with growing number of iterations. Proxies of this error can be monitored, e.g., using analytics derived from the matrix product state approximation 135, and a threshold can be set such that the method stops once the observed measure of error is below the threshold. In particular, the system 100 can calculate various metrics related to the accuracy of the approximation by computing one or more matrix decompositions, e.g., using, one or more of pseudo-matrix inversions, singular value decompositions, e.g., a decomposition of a matrix into a first rotation, scaling, and second rotation matrices, or QR decomposition, e.g., a decomposition of a matrix into an orthonormal matrix and upper triangular matrix. As another example, the system 100 can operate under a timeout restriction. In particular, the system 100 can refine the grid for no more than 20 seconds, 3 minutes, or an hour using the tensor network approximation engine 120.

At the completion of the number of iterations, the tensor network approximation engine 120 can cease updating the adaptive grid and return a final tensor network approximation 170, which can be used to compute the partition function 175. In particular, this can involve deriving the partition function 175 as the normalization constant of the final tensor network approximation 170 of the high-dimensional probability distribution. More specifically, in the case of a Boltzmann distribution, the system 100 can integrate, e.g., sum, each of the Boltzmann factors for all possible states to yield the partition function 175.

The system 100 can additionally derive one or more thermodynamic characterization entities 180 from the approximated partition function 175, e.g., based on a closed-form equation for the partition function Z=∫drdp e^{-E(r,p)/kT}, where the r includes the position vectors of the atoms of the molecular system, p is a momentum vector for the atoms of the molecular systems, k is the Boltzmann constant, and T is a temperature. Furthermore, the final tensor network approximation 170 can be used to calculate marginal and moment distributions 185, e.g., by integrating over each respective degree of freedom to provide a distribution of the states over the remaining degrees of freedom. In particular, once the partition function approximation 175 is known, the probability of observing a given degree of freedom, e.g., based on a specific molecular conformation, can be computed using the standard formula

$$P ( r , p ) = \frac{{e}^{- E ( r , p ) / kT}}{Z} .$$

The marginal and moment distributions 185 can be used for computation of potential of mean force (PMF), free energies of binding or of conformations, and in general any thermodynamic or other quantity which depends on these degrees of freedom.

As a particular example of a thermodynamic characterization entity 180, the partition function approximation 170 can be used to calculate the free energy of the system, e.g., the capacity of the molecular system to do work or expend energy at a constant temperature. The partition function approximation 180 can be used to define the free energy landscape 190, e.g., the probability distribution of the free energy at different temperatures, and to evaluate the free energy at different temperatures T of the system: F=−KT ln Z, where k is the Boltzmann constant and Z is the partition function. In particular, the free energy landscape 190 can be used to understand the free energy of binding, the free energy of solvation (e.g., by running a free energy perturbation calculation and comparing results for a ligand in solution and a ligand in vacuum), and protein folding.

As an example, the system 100 can sample poses of a small molecule, e.g., a drug, in a given active site of another molecule, e.g., a protein target, using the molecular configuration sampler 140. The resultant free energy landscape 190 can be used to approximate the binding affinity between the drug and protein target. In particular, using the final tensor network approximation 170 of the Boltzmann distribution, the system 100 can approximate the binding free energy landscape, which allows for a prediction of drug activity with a target protein, e.g., how effective a given drug will be at treating a disease associated with the target protein. More specifically, the free energy landscape 190 can inform affinity prediction in the hit-to-lead and lead-optimization stages of drug discovery as described, as well as other areas, such as materials science and chemistry, where the free energy landscape 190 can be used to inform the design of catalysts, active materials such as solar panels, and molecules useful for PFAS (“forever chemicals”) removal, etc.

As another example, the system 100 can compute the free energy of solvation associated with a given molecule, e.g., a drug, and a given substrate, e.g., water. More specifically, the free energy landscape 190 can be used to predict the solubility, e.g., tendency of the molecule to dissolve into the substrate, which can provide useful insight in many chemistry and material science applications. For example, the free energy of solvation can inform drug discovery by increasing understanding of the absorption, distribution, metabolism, and excretion of a drug as well as other toxic properties.

As a further example, the system 100 can compute the free energy of interaction associated with a given repeated pattern, e.g., a crystal structure, of a molecule. More specifically, the free energy landscape 190 can be used to predict the most likely arrangement of a given molecule in its solid state. For example, the free energy of interaction can inform material science and chemistry applications of new materials, alloys, etc. and drug design, e.g., the practicality of manufacturing a drug as a pill.

As yet another example, the system 100 can be used to predict the experimentally observed states of molecules under different conditions. In particular, the binding free energy landscape 190 can be used to inform the predicted structure of a large, flexible molecule, e.g., a protein, ribonucleic acid (RNA), deoxyribonucleic acid (DNA), or inorganic polymer, under a variety of conditions, such as different temperatures or in response to an interaction with another molecule, e.g., during a binding interaction. For example, the binding free energy landscape 190 can be used to predict the folded and dynamic states of proteins without requiring a large database of training examples. As another example, the binding free energy landscape 190 can be used to understand potential applications of inorganic polymers.

FIG. 2 illustrates a matrix product state approximation of a full tensor. While the non-lattice partition function approximation system of FIG. 1 does not typically calculate or store the full tensor in a computer-readable memory device, FIG. 2 provides a useful visual of unrolling a full tensor to demonstrate how a matrix product state can be employed as a low-rank approximation of the higher-dimensional tensor object, e.g., to decrease resources required to store and interact with the tensor on a computer readable memory device.

In the particular example depicted, the full tensor 200 has seven “legs”, each representing a dimension of the tensor. In an example of a real molecular system, the tensor can have 20, 30, or 70 legs. As an example, each leg can correspond to a different degree of freedom, e.g., as defined by the one or more molecular degrees of freedom included in the thermodynamic entities 110 of FIG. 1. In this case, each leg represents an indexable axis of variation for the tensor, e.g., a dimension of the tensor that can be traversed by manipulating the value of the degree of freedom along the axis.

The full tensor 200 can be decomposed along each of the legs to form the low-rank matrix product state approximation 240, e.g., an approximation of the full tensor 200 based on a subset, e.g., a few entries of it. In particular, the tensor can be “unrolled” using a bipartition, e.g., by separating nonoverlapping degrees of freedom. More specifically, each dimension or index that corresponds with a molecular degree of freedom can be separated out as a set of one or more matrices that can be multiplied together to reconstruct the full tensor.

In some cases, singular value decomposition (SVD) can be used to separate the dimensions The successive steps of unrolling depict an input x1 205 being separated from the full tensor 200, an input x2 215 being separated from the unrolled tensor 1 210, and an input x3 225 being separated from the unrolled tensor 2 220, leaving behind the unrolled tensor 3 230. In this case, each input corresponds to a particular degree of freedom. Each circle of a respective input represents the set of one or more matrices as a result of the decomposition, e.g., using SVD. Four more successive unrolling steps create the matrix product state approximation 240 over the seven degrees of freedom, represented by separated legs.

In particular, the matrix product state approximation 240 contains seven indexed sets of matrices, e.g., one set of indexed matrices for each molecular degree of freedom, that can be combined, e.g., contracted along all indices, using a matrix product to recover the full tensor. In some cases, the matrix product of all the sets of matrices can be restricted to provide a scalar value, e.g., the first set of matrices can be defined to be a set of single rows and the last set of matrices can be defined to be a set of single columns. In other cases, the trace can be taken of the matrix product of all the sets to provide a scalar value.

In particular, the system, e.g., the non-lattice partition function approximation system 100, can use the matrix product state approximation 240 to provide an efficient means of storing the tensor network approximation and accessing the value of a given multi-index, e.g., a pivot index, using a matrix product. More specifically, storing the full tensor 200 requires an amount of information, e.g., information stored in computer memory, that scales exponentially with the number of legs, e.g., degrees of freedom, but storing the matrix product state approximation 240 requires an amount of information that scales linearly with the number of legs, e.g., degrees of freedom. As a result, the approximation can offer an exponential reduction of computational complexity for evaluating the energy or the probability of a particular multi-index.

Furthermore, using a matrix product state approximation 240 to approximate a non-separable probability distribution allows for other advantages. As an example, the matrix product state approximation 240 can be configured to provide normalized probabilities. As another example, the reconstruction of the full tensor 200 provided by the matrix product state approximation 240 has no statistical error. As yet another example, it is possible to generate unbiased samples from the approximate probability distribution.

FIG. 3 demonstrates an overview of the application of a tensor-train cross approximation (TTCA) algorithm that can be used to construct the tensor network approximation of multivariate probability distribution, e.g., the Boltzmann function. An example of a specific implementation of the TTCA algorithm can be found in Dolgov, S., Anaya-Izquierdo, K., Fox, C. et al. “Approximation and sampling of multivariate probability distributions in the tensor train decomposition”, Stat Comput 30, 603-625 (2019), https://doi.org/10.1007/s11222-019-09910-z. In particular, the tensor network approximation engine 120 of FIG. 1 can use TTCA to construct the tensor network approximation 130 of FIG. 1.

The way in which TTCA specifies the construction of the approximation, e.g., the approximation of the full tensor partition function, is interleaved with the functioning of the adaptive mesh constructor, e.g., the adaptive mesh constructor 150 of FIG. 1. In particular, TTCA involves choosing the multi-indices as pivot indices for evaluation, thereby constructing an optimally efficient grid of unevaluated molecular configuration states. In particular, TTCA constructs an approximation of a given array, e.g., a tensor, by defining a set of rows and a set of columns that can be contracted to recover the full tensor, freezing all but two index dimensions of the tensor, and varying the values of the index to determine whether to add new rows to the set of rows or whether to add new columns to the set of columns that are used to provide the tensor approximation.

In particular, the system can use TTCA to iteratively choose combinations of two unfrozen index dimensions over the course of constructing the tensor network approximation. More specifically, the system can define a tensor bipartition each time the two unfrozen index dimensions are chosen, e.g., certain values of the probability function can become fixed with respect to the determined frozen indices. In some examples, the bipartition can include two subsets of the full tensor, e.g., as defined by a bipartition that separates non-overlapping degrees of freedom. In some cases, the two unfrozen indices can be chosen with respect to the molecular degrees of freedom of the legs of the tensor. For example, in the case of the matrix product state representation of FIG. 2, the system can evaluate combinations of the degrees of freedom in a set order, e.g., from left to right, for each of the indices represented by the legs and then repeat for a set number of iterations. In some examples, the set number of iterations can be parametrized in terms of a multiple of the number of degrees of freedom. The quality of the tensor network approximation as constructed with TTCA is determined by the choice and the number of pivots, as will be described below.

A cross-interpolation, e.g. the cross-interpolation A 300 and the cross-interpolation B 350, refers to interpolating a tensor, e.g., the full tensor 200, using only evaluations of the tensor entries along a subset of all possible multi-indices for a given partitioning. For example, given a tensor Tᵢ_{₁} . . . i_{N} of order N with indices {i₁, . . . , i_{N}}, and a partitioning of the indices into the two disjoint sets {i₁ . . . iₖ}, {iₖ₊₁, . . . , i_{N}}, multi-indices i_{<k}=(i₁, . . . , iₖ) and i_{>k}=(iₖ₊₁, . . . , i_{N}) can be defined. For the given multi-indices, the tensor can be folded into a matrix, e.g., transformed into a lower-dimensional tensor that preserves the data in the full tensor, T₍ᵢ_{₁} _{. . . i}_{ₖ}_{),(i}_{ₖ₊₁} _{. . . i}_{_{N}}₎. For the given folding, the full matrix can be approximated by a skeleton approximation, e.g., a matrix product of three matrices which are computed from a subset of row and column indices of the full matrix. In particular, the identified multi-indices can intersect in crosses, and the approximation is exact at these intersection points.

Importantly, for a rank R matrix, the approximation becomes exact if one chooses R linearly independent rows and columns for the approximation. For a given rank R matrix, an optimal approximation rank P less than R can be determined from maximizing the volume of the P×P submatrix of intersection points, as given by the absolute value of the submatrix determinant. The TTCA implementation of this specification uses a heuristic algorithm to find row and column multi-indices such that the resulting submatrix has a large-volume, e.g., as determined by the submatrix determinant. More specifically, for each pair of neighboring indices iₖ, iₖ₊₁ of a tensor, and a given set of nested pivots {i_{<k-1}}, {i_{>k+1}} the algorithm can: (i) sample a set of k random pivots (i_{<k-1}, iₖ, iₖ₊₁, i_{>k+1}) and (ii) choose the values of iₖ and iₖ₊₁ such that the TTCA at the resulting multi-index has as large a deviation as possible from the exact value. More details on one embodiment of a TTCA approach are included in Sergey Dolgov, Dmitry Savostyanov, “Parallel cross-interpolation for high-precision calculation of high-dimensional integrals.”, Computer Physics Communications, Volume 246, 2020, 106869, ISSN 0010-4655, https://doi.org/10.1016/j.cpc.2019.106869.

In the particular example depicted in cross-interpolation A 300, the unfrozen indices include an unfrozen index A 330 and an unfrozen index B 340. The system can vary the values of the unfrozen index A 330, e.g., continuously along the dimension represented by index A 330. In some cases, the way in which the unfrozen index A 330 is varied involves sampling a random set of values along continuous allowable values, e.g., along the axis corresponding with the particular degree of freedom of unfrozen index A 330. In other cases, the way in which the unfrozen index A 330 is varied is restricted by the allowable values provided by the current gridding defined for the index, as will be covered in greater detail below.

The system can vary the values of the unfrozen index B 340 similarly. In particular, unfrozen index B 340 can be held constant, constant values for the frozen indices can be provided, and the values of unfrozen index A 330 can be evaluated in accordance with a random sample, e.g., to see how the approximation of the probability density changes with respect to the actual value n at the particular multi-index. Then, the system can choose the worst performing value, e.g., the worst performing approximation, in the sample for unfrozen index A 330 and begin to vary unfrozen index B 340. Similarly, unfrozen index A 330 can be held constant, e.g., at the worst performing value, constant values for the frozen indices can be provided, and the values of unfrozen index B 340 can be evaluated in accordance with a random sample, e.g., to see how the approximation of the partition function changes with respect to the actual state evaluation at the particular multi-index.

For the case in which the system can sample a random set of values along continuous allowable values, the grid can be extended to the continuous values sampled. More specifically, extending the grid of the unfrozen index involves drawing samples from the continuous domain of the unfrozen index and comparing the predicted values to the evaluated probability function for all drawn samples. In particular, the system can use a multivariate spline interpolation to obtain a prediction on the continuous domain from the discrete tensor network approximation; and the system can identify the corresponding multi-index specified by each sample for evaluation and perform a numerical or analytical computation to yield the evaluated energy value at the multi-index as an exact value for comparison.

The system can determine the sample with the largest deviation from the directly evaluated values of the probability function as a pivot index. The pivot index takes on the form (i, xₛ, yₛ, l), where i,l are integers, and xₛ,yₛ are continuous values. For the given pivot, the method can determine the boundary pivots (i, j₁, k₁, j), (i, j₂, k₁, j), (i, j₁, k₂, j), (i, j₂, k₂, j), with jₙ, kₘ integers such that x₁=x(j₁)≤xₛ≤x(j₂), y₁=y(k₁)≤yₛ≤y(k₂). In this notation jₙ and kₘ points index the existing grid of the unfrozen variables x and y, and the notation x(jₙ) denotes the value of the unfrozen variable x as provided by the nearest evaluated gridpoint jₙ and y(kₙ) denotes the value of the unfrozen variable y as provided by the nearest evaluated gridpoint kₙ.

Once the boundary pivots are determined, the method can compare the predictions of the tensor network approximation on the boundary pivots e.g., by contracting corresponding indices of the matrix product state approximation to yield the predicted energy value, with the evaluated values of the probability function, e.g., by performing an analytical or numerical computation of the probability function at the specific degree of freedom configuration specified by the corresponding indices. If the prediction error on any of the points is above the error tolerance, the system can incorporate the pivot with the largest error into the tensor-train cross approximation and resume to iterate with a new pair of unfrozen variables. If the error on all four boundary pivots is below the error tolerance, but the error on point (i,xₛ,yₛ,l) is above the threshold, new grid points jₛ and kₛ can be inserted into the existing grid of variables x,y at values such that xₛ=x(jₛ), yₛ=y(kₛ).

The values of the unfrozen indices can be alternately varied as described above to find a multi-index as a pivot index, e.g., an index pair that includes an index value for unfrozen index A 330 and an index value for unfrozen index B 340. In some examples, a pair of pivot indices can be defined for each degree of freedom associated with the first and second unfrozen indices. In particular, a pair of pivot indices can parametrize a new placement of the grid of unevaluated values by defining a boundary between evaluated and unevaluated values, e.g., by specifying a lower and upper bound, e.g., using the boundary pivots described above, for the unfrozen index A 330 and the unfrozen index B 340.

As an example, the pivot indices can be determined based on maximizing a certain criterion, such as a volume criterion. In particular, the intersections defined by the pivot-indices can be chosen to maximize the volume of the intersection submatrix defined by the cross, e.g., the intersection submatrix 315 defined by the set of columns 310 and the set of rows 320 of the cross-interpolation A 300. In some cases, the volume of the submatrix 315 can be defined based on a measure of the determinant of the submatrix. As an example, the volume of the submatrix 315 can be calculated as the absolute value of the determinant of the submatrix: vol A=|det(A)|, where A is the intersection submatrix 315.

The system can maximize the volume, e.g., the volume the submatrix 315, by alternatively updating the set of rows 320 by varying one unfrozen index, e.g., unfrozen index B 340, and the set of columns 330 by varying the other unfrozen index, e.g., unfrozen index A 330. As an example, the members of the sets of evaluated energy state values in the set of rows 320 or the values in the set of columns 330 can be increased by adding one column or row at a time, respectively, e.g., using rook pivoting. In particular, a rook pivot move will increase the size of the submatrix 315 by one dimension, in either the row or column direction, hence obtaining a rank approximation that includes one more dimension with respect to the previous rank approximation. More specifically, the system can choose the additional row or column to increase the volume of the new submatrix as much as possible, e.g., as compared to other possible choices.

More specifically, the system can evaluate the intersection matrix 315 represented by the pivot index of a given cross based on the given value of the first unfrozen index A 320 and the given value of the second unfrozen index B 330, e.g., as the system alternately varies the random set of samples along the first unfrozen index A 320 and the second unfrozen index B 330 along each respective degree of freedom dimension. In particular, the system can evaluate the volume of the intersection matrix 315 at a set cadence, e.g., every N iterations of variation, e.g., one variation of the first unfrozen index A 320 or one variation of the second unfrozen index B 330, or at the completion of one combined variation of the first unfrozen index A 320 and the second unfrozen index B 330.

Likewise, for cross-interpolation B 350, the system can freeze all but two indices, e.g., the unfrozen index A 380 and the unfrozen index B 390, and iteratively and alternatively randomly sample values along each unfrozen index. In particular, unfrozen index B 390 can be held constant, constant values for the frozen indices can be provided, and the values of unfrozen index A 380 can be evaluated in accordance with a random sample, e.g., to see how the approximation of the probability function changes with respect to the actual probability state evaluation at the particular multi-index. Then, the system can choose the worst performing value, e.g., the worst performing approximation, in the sample for unfrozen index A 380 and begin to vary unfrozen index B 390. The cycle of iterating between unfrozen index A 380 and unfrozen index B 390 can continue until indices are chosen to maximize the volume of the intersection submatrix 375 defined by the cross in accordance with the set of columns 360 and the set of rows 370 of the cross-interpolation B 350.

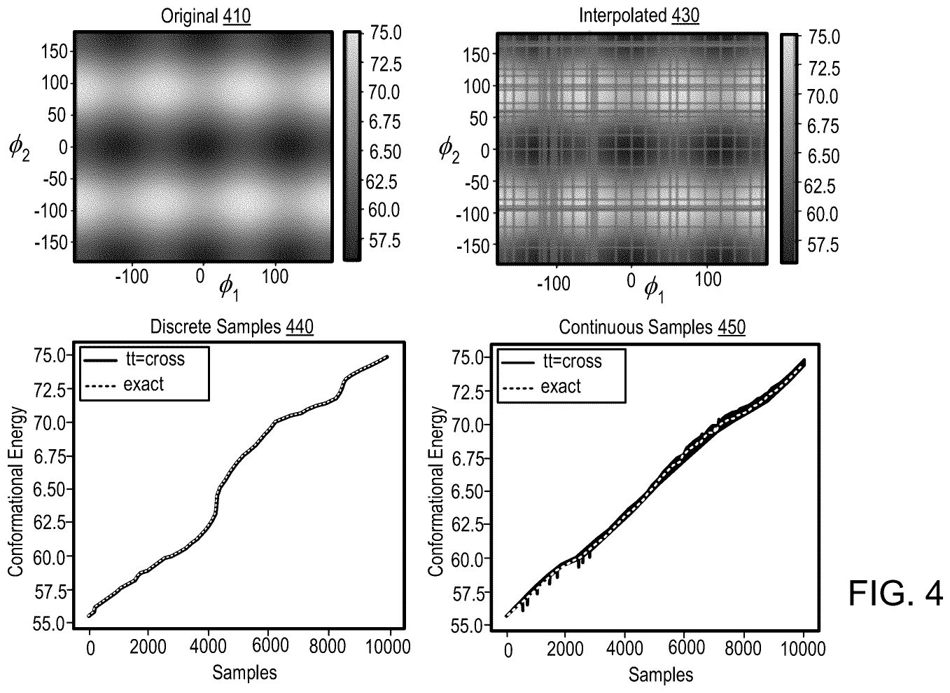

FIG. 4 demonstrates a comparison between energy observation results from an approximated probability function calculated by a continuous molecular dynamics simulation and an approximated probability function approximated using the system described, e.g., the non-lattice partition function approximation system 100 of FIG. 1. In particular, the system can use the adaptive mesh constructor to strategically evaluate the probability function in order to efficiently evaluate and provide the tensor network approximation, e.g., using a tensor train cross approximation algorithm illustrated in FIG. 3.

The particular example depicted is for approximating the conformational energy for an example molecule. For example, the system can receive thermodynamic entities including one or more molecular degrees of freedom, an electric force-field function, particle masses, and a temperature of the molecule. In particular, instead of one or more Cartesian coordinates defining the degrees of freedom, the system can receive one or more molecular degrees of freedom parametrized by angles and dihedrals, e.g., an angle formed by two plane faces, as degrees of freedom.

Plots 410 and 430 depict the conformational energy for the molecule sampled over different configurations of a molecule with two degrees of freedom, e.g., the ϕ₁ and ϕ₂ as degrees of freedom. As an example, the degrees of freedom ϕ₁ and ϕ₂ can be two dihedral degrees of freedom that parametrize two rotatable bonds in the molecule, e.g., ϕ₁ can be the angle of rotation for a first bond and ϕ₂ can be the angle of rotation for a second bond.

The plots 410 and 430 illustrate the conformational energy using a spectrum, e.g., the brighter a given point defined by a particular value of ϕ₁ and a particular value of ϕ₂ is, the more conformational energy the configuration has. In particular, the molecular configuration sampler 140 can sample the energies over different configurations of the two dihedral degrees of freedom using the tensor-train cross approximation algorithm discussed in FIG. 3 to approximate the energy probability for states of the different conformations. Then, the tensor-train cross approximation can be used to evaluate the states of different configurations based on specified values of the two dihedral degrees of freedom.

More specifically, the interpolated approximation of graph 430 illustrates the accuracy of energy states observed from the tensor network approximation with respect to the continuous molecular dynamics simulation of graph 410. As an example, the continuous molecular dynamics simulation needs to be run on the order of 10,000 to 1,000,000 times to provide an approximation of the conformational potential energy as a continuous distribution, e.g., the graph 410, over the two degrees of freedom of the molecule. In contrast, the tensor approximation of graph 430 can be evaluated with a factor of the number of degrees of freedom times less iterations, e.g., 100 times less iterations for a system with two degrees of freedom, than the molecular dynamics simulation of graph 410 and still provide a high-fidelity approximation of the energy landscape, as shown in the graphs 440 and 450, which are described below. In particular, the tensor approximation can allow for approximating the probability function at polynomial cost in system size, e.g., as opposed to exponential cost, thereby enabling a polynomial-efficient calculation of the partition function relative to the continuous molecular dynamics simulation.

As discussed, the lines, e.g., the grid of conformational potential energy graph 430, represent points where the probability function is not evaluated using the molecular dynamics simulation, e.g., places where the partition is interpolated from the known values of the tensor network approximation. In the particular example depicted in graph 430, the grid was adaptively chosen by the tensor-train cross approximation algorithm discussed in FIG. 3.

Furthermore, the sampling graphs 440 and 450 illustrate the sampling of energy state observations from the tensor network approximation. In this particular example, the sampling of the adaptive mesh grid was performed by sampling pivot indices for incorporation into the evaluated tensor network discretely on the grid, e.g., sampling using the discrete gridding values corresponding with the molecular degrees of freedom variables on the plane defined by the two unfrozen degrees of freedom.

The graph 440 illustrates the accuracy of the tensor network by providing results of discrete sampling, e.g., sampling partition function values that are provided exactly by the tensor network approximation, e.g., as provided by the evaluated values specified by the identified pivot indices. In particular, the discrete probability samples fall on the exact line of the conformational energy function, e.g., the sampled indices were evaluated exactly using the energy function and therefore provide exact values of the partition function at the pivot points.

The graph 450 illustrates the accuracy of the tensor network by providing results of continuous sampling, e.g., continuous sampling that includes interpolated partition function values. In particular, the continuous energy observation samples fall on the exact line of the conformational energy function when the sample corresponds with evaluated values of the probability function and fall close to the exact line when the sample corresponds with values interpolated from the exactly evaluated values of the tensor network. The limited deviation of the continuous samples from the exact line demonstrates the accuracy of the approximation of the tensor network, e.g., the interpolation of the tensor network provides an effective approximation of the energy states at the unevaluated indices of the adaptive mesh grid.

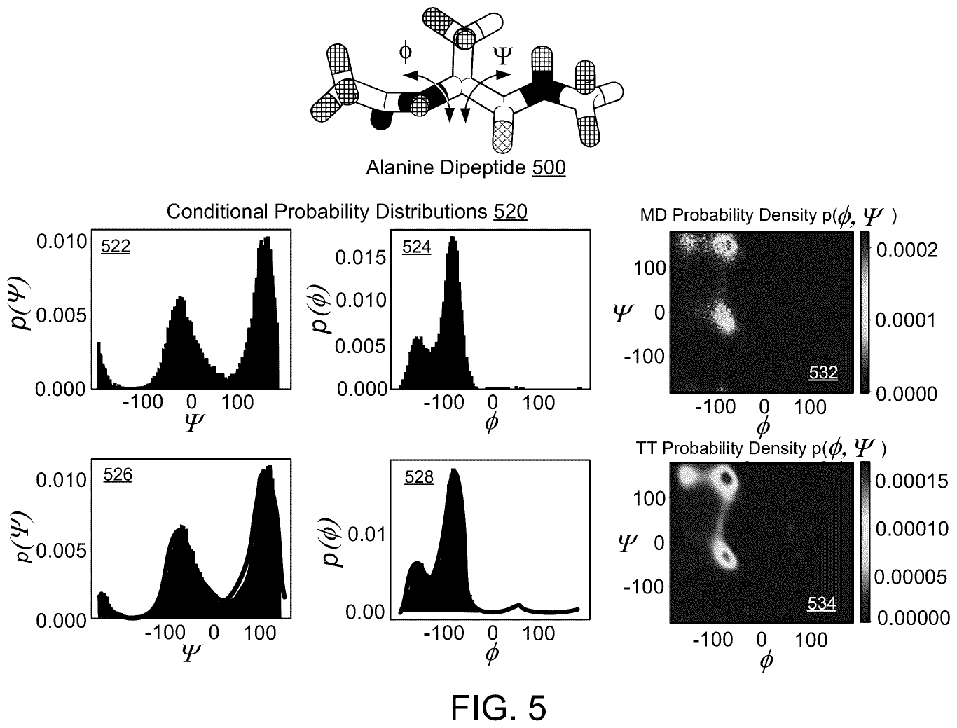

FIG. 5 provides example results of the approximated partition function e.g., a probability density function over potential energy states, and the approximated marginal distribution, over each degree of freedom, for alanine dipeptide using a classical molecular dynamics (MD) approximation and a non-lattice partition function approximation system, e.g., the non-lattice partition function approximation system 100 of FIG. 1.

As an example, the MD simulation can involve integrating physics equations governing the atoms of the alanine dipeptide molecule forward in time to find the most likely energy configurations. MD simulations avoid calculating the partition function directly, and instead rely on assessing the probability of different configurations of the molecular system. However, calculating the MD simulation is slow and uniform convergence of the calculated function is not guaranteed.

Alanine dipeptide 500 is a standard benchmark molecule for living systems, and, in particular, for modeling different dihedral angle conformations in proteins. The Ramaschandran angles, labeled phi and psi in the diagram of alanine dipeptide 500, are defined by four points in space and correspond with two planes intersecting at different angles. The Ramaschandran angles are soft degrees of freedom that specify potential torsions of the molecule.

In the particular example depicted, the marginal probability distributions 520 for the torsional phi and psi angles as modeled by the classical MD simulation are displayed in graphs 522 and 524 and as modeled by the tensor network approximation are displayed in graphs 526 and 528. Despite being evaluated with only a fraction of calls to evaluate the probability function, e.g., on the order of 10,000 for the tensor network approximation compared to millions for the MD simulation, the marginal probability distributions, which integrate over the probability function, in graphs 526 and 528 reproduce the marginal probability distributions in graphs 522 and 524 with fidelity, e.g., with a relative error of less than 1E-2.

The probability density plots 532 and 534 further depict the results of the approximated partition function for the MD simulation and tensor train cross approximation, respectively. In particular, the partition function approximated by the tensor-train cross approximation produces the TT probability density in graph 534, which is smoother than the partition function approximated by the MD simulation in graph 532, e.g., because of interpolation of the unevaluated values on the grid. Additionally, as mentioned previously, the tensor network approximation is produced with less compute than required to produce the partition function approximation with the MD simulation. More specifically, the approximation of the tensor-train cross approximation scales linearly with the number of degrees of freedom, as opposed to exponentially with respect to the MD simulation.

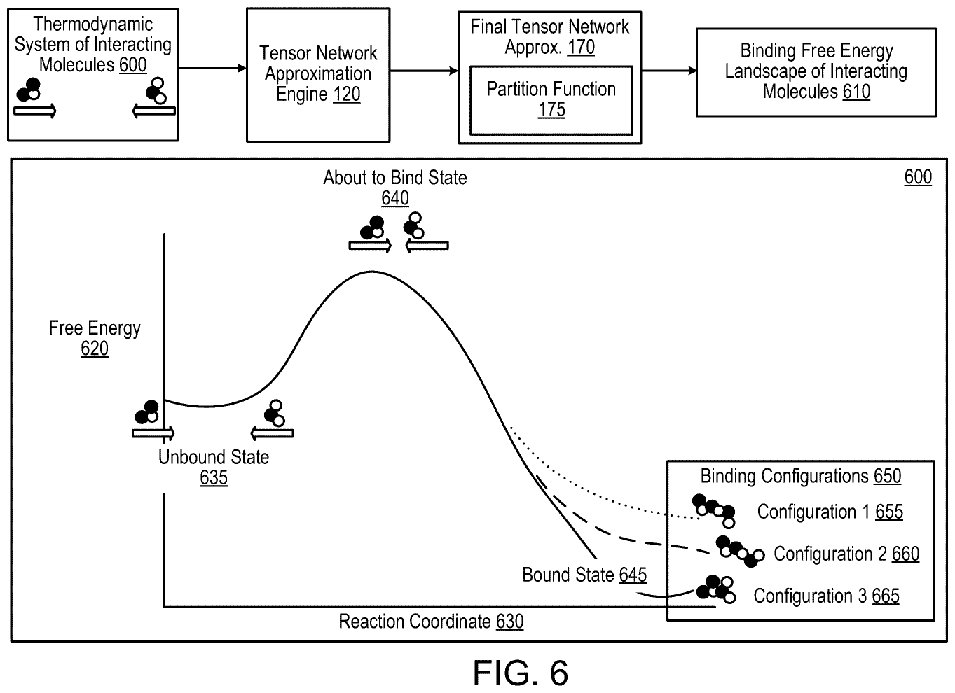

FIG. 6 demonstrates how an approximated partition function for a non-lattice molecular system including a small and large molecule can be used to assess the binding free energy landscape, e.g., as part of a drug discovery process. In particular the non-lattice partition function approximation system 100 of FIG. 1 can be used to provide the binding free energy landscape for the interacting small and large molecule system 610, e.g., a protein and ligand or genomic state and drug, which can be used for downstream tasks in drug discovery.

As described in FIG. 1, the system can process thermodynamic entities regarding a system of interacting molecules 600 using a tensor network approximation engine 120 to provide the partition function 175, e.g., by integrating over the states in the final tensor network approximation 170. As an example, this can involve placing the small molecule, e.g., drug or ligand, at the binding site using a standard docking approach. As an example, scoring functions, genetic algorithms, or Monte Carlo can be used to predict the ligand or drug-receptor complex structure. For example, Local Move Monte Carlo sampling, e.g., as detailed in Meng X Y, Zhang H X, Mezei M, Cui M, “Molecular docking: a powerful approach for structure-based drug discovery”, Current Computer-Aided Drug Design 2011 June Volume 7 Issue 2 pg. 146-57, DOI: 10.2174/157340911795677602, can be used to simulate molecular docking. In other words, Local Move Monte Carlo Sampling can be used to simulate the way in which a large molecule's configuration changes to accommodate the binding of a small molecule.

The interaction between the small and large molecule is represented in the reaction coordinate plot 600, which provides the free energy 620, e.g., the capacity of the molecular system to do work or expend energy at a constant temperature, as a function of the reaction coordinate 630, e.g., a representation of progress along a reaction pathway for two binding molecules. In particular, the binding between the two interacting molecules system can involve two energy minima: one for when the two molecules are far away, e.g., in an unbound state 635, and one for when they are bound together in a bound state 645, e.g., in one of the possible binding configurations 650. The reaction coordinate plot 600, demonstrates how the two molecules approach from the unbound state 635 to an intermediate state, e.g., the about to bind state 640, which temporarily increases the energy, before binding in the bound state 645, e.g., in one of binding configuration 1 655, configuration 2 660, or configuration 3 665.

In particular, the binding configurations 650 lower the energy of the small and large molecule system with respect to both the unbound state 635 and the about to bind state 640, as evident from the sharp decrease of the free energy in the graph 600 in the bound state 645. However, there are several possible binding configurations 650 that lower the energy. In this particular example, binding configuration 1 655, binding configuration 2 660, and binding configuration 3 665 are potential bound states 645. In more realistic examples, there can be hundreds or thousands of potential binding configurations 650 for an interacting molecule system.

In the particular example depicted, the binding free energy landscape of interacting molecules 610 is determined by the partition function 175. In this case, the free energy of binding is an integral over the probability distribution, e.g., the free energy of configuration 1 655, the free energy of binding configuration 2 660, and the free energy of binding configuration 3 665 can be obtained using the partition function 175: F=−KT ln Z. In particular, the partition function 175 can be used to calculate the change in free energy, e.g., by comparing the free energy in the unbound state 635 with the free energy in the bound state 645:

$$Δ F = - k T l n \frac{{Z}_{b o u n d}}{{Z}_{u n b o u n d}} .$$

The binding free energy landscape of interacting molecules 610 can be used as part of a larger workflow featuring a model to systematically assess the docking of small molecules, e.g., drugs, with a target protein. The binding free energy landscape 610 can describe the distribution of possible docking configurations along with the probability and likelihood of the docking configuration appearing for an interaction between a protein and small molecule. In particular, different drug molecules can be assessed from a library of known molecules and the thermodynamic system of interacting molecules 600 can be processed by the system to find the one or more positions of the drug interacting with the protein. As an example, if a protein is known as a target, e.g., symptomatic of a disease, the binding free energy landscape of interacting molecules 610 can be approximated using the tensor network approximation 120 and used to select a drug that binds to the given protein target based on a set of criteria, e.g., a set of activity criteria.

As an example, the system can approximate the binding free energy landscape 610 of the interacting system using TTCA to compute a docking score, e.g., a score based on the free energy of the binding configuration, for different binding configurations 650. In particular, computing a docking score directly from the partition function 175 can provide a more accurate way of assessing binding configurations. In particular, docking scores can be used to rank binding configurations, e.g., the binding configurations 650. More specifically, calculating a system characterization quantity from the partition function, e.g., free energy perturbation scores that measure a discrepancy between two states, e.g., the unbound state 635 or the about to bind state 640 and one of the possible bound states 645, is a more sophisticated way of determining docking scores. Furthermore, the computation of a docking score from the final tensor network approximation 170 can be performed within minutes given a single modern GPU core. Hence, the approach is scalable to millions of candidate drugs by employing cloud-based computing architectures on a large scale.

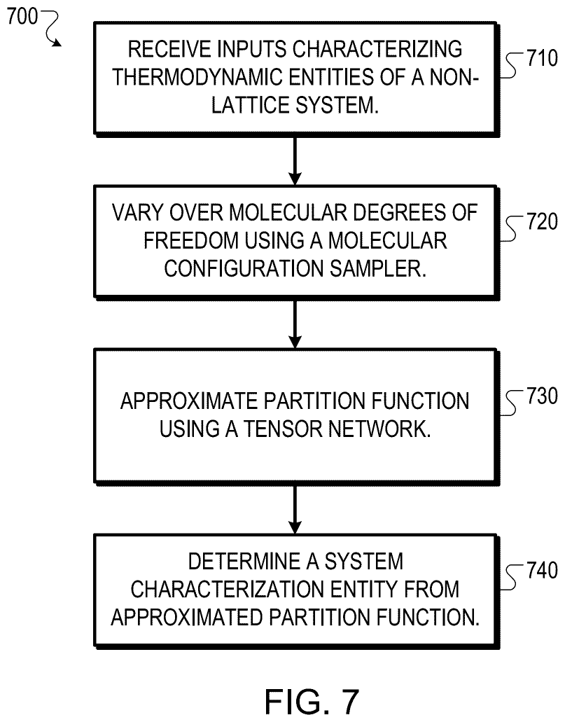

FIG. 7 is a flow diagram of operations for approximating a partition function of a non-lattice molecular system of one or more molecules that can be used to determine a system characterization entity. For convenience, the process 700 will be described as being performed by a system of one or more computers located in one or more locations. For example, a non-lattice partition function approximation system, e.g., the non-lattice partition function approximation system 100 of FIG. 1, appropriately programmed in accordance with this specification, can perform the process 700.

In particular, the system can receive inputs characterizing the thermodynamic entities of a non-lattice system (step 710), e.g., a non-lattice molecular system of one or more molecules. In particular, a non-lattice molecular system can be a molecular living system, e.g., a protein or a small molecule, or non-living system, e.g., an inorganic polymer. For example, the set of inputs can include one or more of one or more molecular degrees of freedom, a force field function, particle masses, and a temperature.

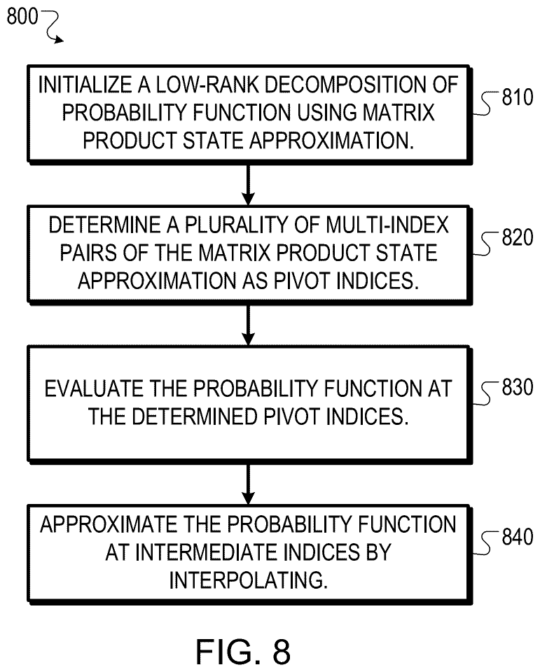

The system can vary over the molecular degrees of freedom, e.g., the molecular degrees of freedom from the inputs in step 710, using a molecular configuration sampler (step 720). In particular, the system can vary over the molecular degrees of freedom to provide and assess the energy distribution of different conformational states, e.g., different shapes or structures of the one or more molecules. The system can approximate the partition function of the non-lattice system using a tensor network (step 730), e.g., by evaluating a high-dimensional probability function, e.g., the Boltzmann distribution, at the different molecular degrees of freedom provided by the molecular configuration sampler and integrating over the approximated distribution. As an example, the system can use a method to evaluate the probability function along the available degrees of freedom, as will be described in further detail in FIG. 8.

The system can determine a system characterization entity or function from the approximated partition function (step 740). In particular, the system can use the probability function to derive marginal and moment distributions or to calculate the probability of observing a particular energy state. As an example, the approximated partition function can be used to assess binding affinities, e.g., for drug discovery, solubility prediction, crystal structure prediction, and/or polymer conformation prediction.

FIG. 8 is a flow diagram of an example process for approximating a partition function using a tensor network, e.g., by strategically sampling from a probability function to approximate a probability function that can be integrated to approximate the partition function. For convenience, the process 800 will be described as being performed by a system of one or more computers located in one or more locations. For example, a tensor network approximation engine 120, e.g., the tensor network approximation engine 120 of FIG. 1, appropriately programmed in accordance with this specification, can perform the process 800.

The system can initialize a low-rank decomposition of the probability function using a matrix product state approximation (step 810). In particular, the use of one or more matrix product states, e.g., smaller tensors that can reconstruct the full tensor when contracted over all indices, compresses the set of values needed to express the probability function. In particular, by representing the approximated probability function using the matrix product state approximation, it is possible for the system to manipulate the one or more matrix product states to modify the tensor network approximation.

The system can determine a plurality of the multi-index pairs of the matrix product state approximation as pivot indices (step 820). In this case, a multi-index provides a location in the tensor, e.g., as specified by one or more index values associated with each respective dimension of the tensor. The system can then evaluate the probability function at the determined pivot indices (step 830). In particular, accessing a multi-index of the one or more matrix product states returns an approximation to the true value of the full tensor at the same multi-index. For example, the system can strategically determine which multi-index pairs to evaluate, e.g., using a tensor-train cross approximation algorithm.

The system can then approximate the probability function at the intermediate indices, e.g., the unevaluated indices, by interpolating (step 840). As an example, the unevaluated indices can be interpolated by fitting a linear, quadratic, or spline approximation between the evaluated values to approximate the unevaluated indices of the probability function. In some cases, the unevaluated indices form a grid. More specifically, the system can adaptively construct a grid of molecular configuration states, e.g., an adaptive mesh grid, by selecting regions of high importance, high variance, or both to the probability function to evaluate exactly, e.g., as determined by the pivot points, and interpolate the other unevaluated values, e.g., the values on the grid.

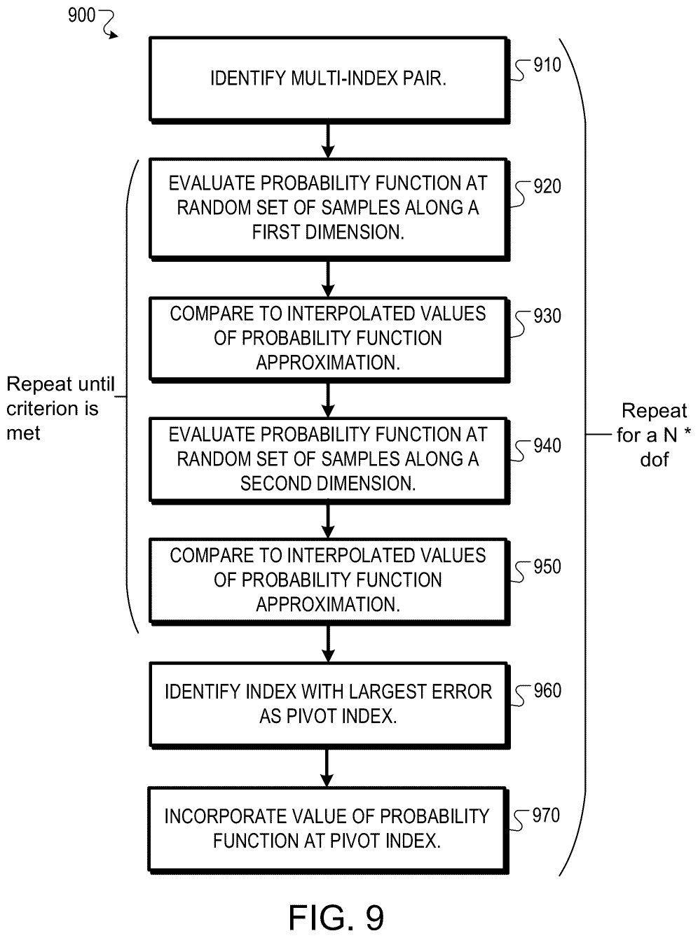

FIG. 9 is a flow diagram of an example process for determining the multi-indices of an adaptive mesh grid, e.g., as part of a tensor-train cross approximation. For convenience, the process 900 will be described as being performed by a system of one or more computers located in one or more locations. For example, a tensor network approximation engine, e.g., the tensor network approximation engine 120 of FIG. 1, appropriately programmed in accordance with this specification, can perform the process 900.

The system can first identify a multi-index pair (step 910). The multi-index pair includes a set of two multi-indices that describe two locations in the full tensor. In particular, the multi-index pair can define a bipartition of the tensor, e.g., two subtensors with nonoverlapping degrees of freedom. In an example with four degrees of freedom, e.g., degrees of freedom parametrized by the x1, x2, x3, and x4 dimension, the multi-index pair can be a set of index values along each of the x1, x2, x3, and x4 dimensions, respectively, e.g., the multi-index pair (a, b, c, d) and (e, f, g, h). More specifically, in this case, a and e can define two values for the x1 degree of freedom, b and f can define two values for the x2 degree of freedom, c and g can define two values for the x3 degree of freedom, and d and h can define two values for the x4 degree of freedom.

The system can then evaluate the probability function at a random set of samples along a first dimension, e.g., a first set of index values out of the x1, x2, x3, and x4 degrees of freedom (step 920). More specifically, the system can freeze the values of all but two of the multi-indices, e.g., x1 and x2, and evaluate random sets of samples along the x1 degree of freedom, e.g., by holding values of x2, x3, and x4 constant while varying the value of the x1 degree of freedom, e.g., (random value of x1, b, c, d), In particular, the system can randomly sample values for the x1 degree of freedom and compute the probability function of the molecular configuration as defined by the sampled index.

In some cases, the system can randomly sample continuous values for each degree of freedom. In other cases, the system can be restricted to sampling discrete values as provided by the allowable gridding of the dimensions. The system can then compare the evaluated energy function with an interpolation provided by the tensor network approximation (step 930), e.g., using a spline interpolation. In particular, the system can assess the discrepancy between the evaluated probability function, e.g., using numerical or analytical computation, and the approximated probability function.

The system can then evaluate the probability function at a random set of samples along a second dimension, e.g., a second set of index values (step 930). More specifically, the system can freeze the values of all but two of the multi-indices, e.g., x1 and x2, and evaluate random sets of samples along the x2 degree of freedom, e.g., by holding values of x1, x3, and x4 constant while varying the value of the x2 degree of freedom, e.g., (a, random value of x3, c, d) in the bipartition. For example, the value of the x1 degree of freedom can be determined to be the index value with the greatest discrepancy between the evaluated probability function and the approximated value provided by the tensor network in step 930. The system can randomly sample values, e.g., continuously or discretely, along the second dimension, e.g., the x2 degree of freedom, and compute the probability function of the molecular configuration as defined by the sampled index. Similarly to step 930, the system can compare the evaluated probability function with an interpolation provided by the tensor network approximation (step 950), e.g., using a spline interpolation, to assess the discrepancy between the evaluated energy function and the approximated energy function.

The system can alternate between varying the first and second dimension by repeating steps 920-950 to adaptively construct a grid over molecular configuration states. For example, the value for the x2 degree of freedom can have been determined to be the index value with the greatest discrepancy between the evaluated probability function and the value provided by the tensor network in step 950 in the next iteration, and the next value for the x1 degree of freedom when varying the value of x2 can be determined by the index value with the greatest discrepancy found in step 930, and so on. This alternating evaluation of a random set of samples along a first and second dimension can continue until a criterion is met.

As an example, the criterion can be related to maximizing a measure of volume of a submatrix defined by the cross-interpolation intersection. Cross-interpolation refers to interpolating the full tensor using only evaluations of the tensor entries along a subset of all possible multi-indices for a given partitioning. In particular, the intersections defined by the pivot-indices can be chosen to maximize the volume of the intersection submatrix defined by the cross-interpolation. In particular, the process can repeat for M iterations, where each iteration increases or maintains the measure of volume of the submatrix. In another case, the criterion can be a timeout criterion. The system can then identify the index with the largest error as the pivot index (step 960). The pivot index can then be added into the tensor network approximation as an evaluated value (step 970).

In the case of random sampling continuously along the first and second dimension, e.g., dimension pertaining to degrees of freedom, the gridding of the tensor network, e.g., the discretization of knowable values along each variable, can change, e.g., be refined to provide a smaller mesh grid, to accommodate the new value. In the case of random sampling performed along the existent gridding of the first and second dimension, the tensor network can retain the same gridding discretization. In this case, the values that are added to the tensor network approximation are removed from the grid of unevaluated values and accommodated into evaluated regions refined by the pivot indices.

The system can perform the process 900 over a number of iterations. In particular, the system can perform the process 900 over a set number of iterations with respect to the number of degrees of freedom in the molecular system, e.g., for a multiple of the number of molecular degrees of freedom, e.g., for a number N multiplied by the number of molecular degrees of freedom. For example, the system can freeze all values in the tensor except for the bipartition defined with a particular choice of a multi-index pair and iteratively choose another combination of two of the available indices as variable degrees of freedom, e.g., the system can compute pairwise updates for neighboring indices, e.g., x1 and x2, x2 and x3, and x3 and x4. In some cases, the system can compute pairwise updates for non-neighboring indices.

This specification uses the term “configured” in connection with systems and computer program components. For a system of one or more computers to be configured to perform particular operations or actions means that the system has installed on its software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
receiving a set of inputs characterizing thermodynamic entities of a non-lattice system with one or more molecular degrees of freedom, wherein each molecular degree of freedom is characterized by a gridding of allowable values;
approximating a partition function of the non-lattice system using a tensor network and based at least in part on the set of inputs, wherein approximating a partition function of the non-lattice system comprises:
using a molecular configuration sampler to vary over the one or more molecular degrees of freedom with respect to the gridding of allowable values;
adaptively updating the gridding of allowable values for one or more degrees of freedom based on an approximation error of the partition function, wherein adaptively updating the gridding of allowable values comprises modifying a number of gridpoints of the gridding of allowable values for the one or more molecular degrees of freedom;
determining a plurality of system characterization entities from the partition function for a corresponding plurality of conformations that are each specified by a respective configuration of molecular degrees of freedom, wherein the system characterization entities comprise corresponding respective measures of free energy for each of the plurality of conformations;
generating a free energy landscape using the respective measures of free energy for each of the plurality of conformations; and
using the free energy landscape to determine one or more metrics of the non-lattice system, wherein the one or more metrics comprise at least one of a free energy of solvation, or observation likelihood of a respective conformation, or a combination.

2. The method of claim 1, wherein the inputs characterizing thermodynamic entities of the non-lattice system comprise one or more of molecular degrees of freedom, an electric force-field function, particle masses, and a temperature of the system.

3. (canceled)

4. The method of claim 1, wherein the non-lattice system comprises a classical molecular system of one or more molecules.

5. The method of claim 1, wherein the molecular configuration sampler performs conformational sampling comprising varying over angular and dihedral rotational degrees of freedom of the non-lattice system.

6. The method of claim 1, wherein approximating the partition function of the system using a tensor network comprises using a tensor-train cross approximation to determine an approximated partition function, wherein the tensor-train cross approximation comprises:
initializing a high-dimensional probability function tensor network by generating a low-rank decomposition of matrix product states based at least in part on the set of inputs, wherein the high-dimensional probability function tensor network is associated with a plurality of dimensions corresponding with each molecular degree of freedom, and wherein each dimension is traversable with a respective index along the respective gridding characterizing the molecular degree of freedom for the dimension;
determining a plurality of pivot indices using rook pivoting and cross interpolation, wherein each pivot index comprising a set of index values for each dimension of the tensor network, and wherein the set of index values have been sampled by adaptively updating the gridding of allowable values for each respective dimension of the tensor network based on the approximation error of the partition function;
evaluating the partition function at the pivot indices;
approximating the high-dimensional probability function at a plurality of unevaluated indices by interpolating between one or more evaluated values of the high-dimensional probability function at pivot indices; and
integrating the approximated high-dimensional probability function to determine the approximated partition function of the system.

7. The method of claim 6, wherein approximating the partition function of the system using a tensor network further comprises performing a regression to minimize an objective to a target function comprising the one or more evaluated values of the high-dimensional probability function at pivot indices.

8. The method of the claim 6, wherein interpolating between the one or more evaluated values of the high-dimensional probability function at pivot indices further comprises constructing splines between the one or more evaluated values of the high-dimensional probability function at pivot indices and the plurality of unevaluated indices on the grid.

9. The method of claim 6, wherein determining a plurality of pivot indices using rook pivoting and cross interpolation further comprises, for each pivot index:
identifying a tensor bipartition by selecting a first and second variable dimension for index traversal;
alternatingly evaluating a random set of samples along the first and second variable dimension within the tensor bipartition, wherein evaluating comprises, at each iteration in a number of iterations:
generating a first evaluation of the high-dimensional probability function at a respective set of index values specified by a first sample in the random set of samples;
determining an approximation error by comparing the first evaluation to a second interpolated evaluation at the respective set of index values comprising interpolating between one or more evaluated values of the high-dimensional probability function at pivot indices at the respective set of index values;
updating the gridding of allowable values for a second sample in the random set of samples in accordance with the approximation error between the first evaluation and the second interpolated evaluation;
identifying the respective set of index values with a largest approximation error between the first evaluation and second evaluation; and
adding the first evaluation at the set of index values with the largest approximation error to the tensor network.

10. The method of claim 9, wherein determining a plurality of pivot indices further comprises determining a sequence of pivot indices in accordance with maximizing a measure of volume of a submatrix.

11. The method of claim 10, wherein maximizing the measure of volume of the submatrix further comprises:
calculating a volume metric for an intersection matrix determined by a pivot index, wherein calculating the volume metric comprises, for each pivot index in the sequence of pivot indices:
evaluating the intersection matrix represented by the pivot index comprising a value of a determinant of the intersection matrix determined by the pivot index;
alternately updating the first and second variable dimension of the pivot index; and
choosing the pivot index associated with a maximal volume intersection matrix.

12. The method of claim 1, wherein determining the plurality of system characterization entities from the partition function for a corresponding plurality of conformations further comprises determining a system characterization derivative function or system characterization quantity from the partition function.

13. The method of claim 12, wherein determining the system characterization derivative function comprises determining one or more of marginal and moment distributions over possible degrees of freedom of the partition function.

14. The method of claim 12, wherein determining the system characterization derivative function comprises determining the corresponding respective measures of free energy for each of the plurality of conformations.

15. The method of the claim 14, wherein the measure of free energy comprises a binding free energy describing a binding free energy for an interaction between a protein and a small molecule, and further comprising:
using the binding free energy to determine a distribution of possible docking configurations as a binding free energy landscape; and
for each possible docking configuration in the binding free energy landscape, determining a docking score comprising a probability of the interaction between the protein and small molecule resulting in the docking configuration.

16. The method of claim 15, further comprising identifying a candidate small molecule for inclusion in a drug based at least on the docking score of the candidate small molecule.

17. The method of claim 15, wherein the one or more metrics further comprise a solubility metric, crystal structure metric, and polymer conformation metric for the small molecule.

18. The method of claim 17, further comprising using the one or more metrics to assess at least one of a measure of manufacturability of a pill that includes the small molecule and a measure of toxicity of administering the pill to a subject.

19. The method of claim 1, further comprising designing an active material based at least on the one or more metrics.

20. The method of claim 19, wherein the active material is a catalyst.

21. A system comprising:
one or more computers and one or more storage devices on which are stored instructions that are operable, when executed by the one or more computers, to cause the one or more computers to perform operations comprising:
receiving a set of inputs characterizing thermodynamic entities of a non-lattice system with one or more molecular degrees of freedom, wherein each molecular degree of freedom is characterized by a gridding of allowable values;
approximating a partition function of the non-lattice system using a tensor network and based at least in part on the set of inputs, wherein approximating a partition function of the non-lattice system comprises:
using a molecular configuration sampler to vary over the one or more molecular degrees of freedom with respect to the gridding of allowable values;
adaptively updating the gridding of allowable values for one or more degrees of freedom based on an approximation error of the partition function, wherein adaptively updating the gridding of allowable values comprises modifying a number of gridpoints of the gridding of allowable values for the one or more molecular degrees of freedom;
determining a plurality of system characterization entities from the partition function for a corresponding plurality of conformations that are each specified by a configuration of molecular degrees of freedom, wherein the system characterization entities comprise corresponding respective measures of free energy for each of the plurality of conformations;
generating a free energy landscape using the respective measures of free energy for each of the plurality of conformations; and
using the free energy landscape to determine one or more metrics of the non-lattice system, wherein the one or more metrics comprise at least one of a free energy of solvation, or observation likelihood of a respective conformation, or a combination.

22. One or more non-transitory computer-readable storage media encoded with instructions that, when executed by one or more computers, cause the one or more computers to perform operations comprising:
receiving a set of inputs characterizing thermodynamic entities of a non-lattice system with one or more molecular degrees of freedom, wherein each molecular degree of freedom is characterized by a gridding of allowable values;
approximating a partition function of the non-lattice system using a tensor network and based at least in part on the set of inputs, wherein approximating a partition function of the non-lattice system comprises:
using a molecular configuration sampler to vary over the one or more molecular degrees of freedom with respect to the gridding of allowable values;
adaptively updating the gridding of allowable values for one or more degrees of freedom based on an approximation error of the partition function, wherein adaptively updating the gridding of allowable values comprises modifying a number of gridpoints of the gridding of allowable values for the one or more molecular degrees of freedom;
determining a plurality of system characterization entities from the partition function for a corresponding plurality of conformations that are each specified by a configuration of molecular degrees of freedom, wherein the system characterization entities comprise corresponding respective measures of free energy for each of the plurality of conformations;
generating a free energy landscape using the respective measures of free energy for each of the plurality of conformations; and
using the free energy landscape to determine one or more metrics of the non-lattice system, wherein the one or more metrics comprise at least one of a free energy of solvation, or observation likelihood of a respective conformation, or a combination.

23. The method of claim 9, wherein updating the gridding of allowable values for the second sample in the random set of samples in accordance with the approximation error between the first evaluation and the second interpolated evaluation comprises:
refining an interval between allowable values of the gridding based on the approximation error; or
maintaining the gridding discretization.
